(19) United States
(12) Patent Application Publication
(10) Pub. No.: US 2025/0170044 A1
(43) Pub. Date: May 29, 2025

(54) MULTI-MODAL HAIR CARE COMPOSITION COMPRISING CATIONIC GUAR BLENDS AND PROCESS FOR PREPARATION THEREOF

(71) Applicant: ISP Investments LLC, Wilmington, DE (US)

(72) Inventors: David C. STREULI, Wayne, NJ (US); Lidia KULCSAR, Flanders, NJ (US); Carolie Claudine ALONSO, Breda (NL); T prene CLEMONS, Maplewood, NJ (US)

(73) Assignee: ISP Investments LLC, Wilmington, DE (US)

(21) Appl. No.: 18/841,361

(22) PCT Filed: Feb. 24, 2023
(86) PCT No.: PCT/US23/13849

§ 371 Date: Aug. 23, 2024

(51) Int. Cl. A61K 8/73; A61K 8/04; A61Q 5/00; A61Q 5/12

(52) U.S. Cl. CPC A61K 8/73; A61K 8/046; A61Q 5/002; A61Q 5/12

(57) ABSTRACT

Provided herein is a multi-modal hair care composition comprising: 1 to 99 wt. % of a cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; and 1 to 99 wt. % of at least one cosmetic ingredient.

### FIELD OF THE INVENTION

The present disclosure relates to a multi-modal hair care composition comprising cationic guar blends, and processes for preparing the same.

### BACKGROUND OF THE INVENTION

Hair is a keratinous filament which is subjected to various repeated stresses including mechanical stress from everyday grooming practices (such as frequent brushing and combing), environmental stress (such as UV exposure), damaging chemical treatments (such as bleaching, coloring) and thermal insult from the use of heat styling tools. The damaging effects of these exposures often manifest several types of damage to the hair fiber including structural degradation of the cortex, lifted cuticles, split ends, and other damaging effects that often result in further injury and hair breakage. Formulators of hair care and hair styling are constantly looking for products with multi-functional benefits that can effectively treat and style hair. Synthetic and nature derived polymers are widely used to achieve the desired sensory attributes to hair care. Hair styling and conditioning products encompass both fully synthetic and nature derived polymers to deliver conditioning and styling benefits. Consumers and formulators are searching for sustainable, environmentally friendly (e.g. biodegradable) and nature derived solutions. Many traditional synthetic hair care and styling polymers do not fit the sustainability criteria put forward by governmental and non-governmental entities. As such, there is increasing pressure on manufacturers of hair care and styling products to provide formulations which meet the expected care and styling benefits, while using nature derived, sustainable, biodegradable ingredients. Due to the increased demand for sustainable ingredients, the hair care industry is transitioning from using synthetic chemicals to biodegradable, nature-derived, plant based chemicals. Polysaccharides are a class of nature-derived polymers with a good sustainability profile, typically used in rinse off formulations, especially shampoos, to provide conditioning and deposition of actives.

U.S. Pat. No. 4,061,602 assigned to American Cyanamid Co. discloses conditioning shampoo composition containing a cationic derivative of a natural gum (such as guar) as the active conditioning agent.

U.S. Pat. No. 11,052,033 assigned to Rhodia Operations discloses personal care composition comprising guar derivative with mean average molecular weight of about 250,000 g/mol to about 600.00 g/mol and Guar hydroxypropyl trimonium chloride having a cationic degree of substitution from about 0.20 to 0.30.

US Patent Application Publication No. 2020-0163861A1 assigned to Rhodia Operations discloses a cationic polygalactomannan containing non-ionic hydroxyalkyl substituents and having a Brookfield RVT viscosity at 25° C. and 20 rpm greater than 700 mPa·s, at a concentration of 1 pbw in water, for instance comprised between 700 and 1,200 mPa·s.

Present inventors attempt to prepare a hair care composition using nature derived and biodegradable cationic guar blends. Accordingly, the present applicant discloses cationic polysaccharides traditionally used in rinse-off formulations, that provide excellent styling and conditioning benefits to hair. By combining cationic polysaccharides with different molecular weights and degree of substitutions, it is possible to customize the styling and care benefits of formulations. The combination of polysaccharides with different chemical properties allows formulators to “dial-in” performance and aesthetic properties important to consumers, such as hair stiffness, conditioning, slip properties, humidity resistance, styleability and durability of style. Further, the cationic polysaccharides also help in mending hair split ends, protection from thermal and mechanical damage, and protection of hair from hot flat ironing up to 450° F.

### SUMMARY OF THE INVENTION

In one aspect, the present application provides a multi-modal hair care composition comprising: (i) 0.1 to 99 wt. % of at least one cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; and (ii) 0.1 to 99 wt. % of at least one at least one cosmetic ingredient.

In another aspect, the present application provides a hair care composition for mending split ends comprising (1) 0.1 to 99 wt. % of at least one cationically modified guar blend comprising: (a) 1 to 99 wt. % of at least one cationically modified guar having weight average molecular weight in the range of from about 50 to 500 KDa, and (b) 1 to 99 wt. % of at least one cationically modified guar having weight average molecular weight in the range of from about 500 to 1500 KDa; and (iii) 0.1 to 99 wt. % of at least one cosmetic ingredient.

In another aspect, the present application provides an aqueous hair mousse composition comprising: (i) 0.1 to 10 wt. % of hair care composition comprising cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa, (ii) 1.0 to 60 wt. % of at least one solvent; (iii) 0.1 to 10 wt. % of at least one cosmetic functional additive; and (iv) 0.1 to 95 wt. % of water.

In another aspect, the present application provides a method for repairing split ends of hair comprising contacting hair with split ends with a multi-modal hair care composition comprising: (i) 0.1 to 99 wt. % of at least one cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; (ii) 0.0 to 99 wt. % of a non-ionic guar; and (iii) 0.1 to 99 wt. % of at least one cosmetic ingredient followed by rinsing the hair with water.

In another aspect, the present application provides a method for protecting hair from thermal and mechanical damage, comprising contacting the hair with a multi-modal hair care composition comprising: (i) 0.1 to 99 wt. % of at least one cationically modified guar blend comprising: (a) 1 to 99 wt. % of at least one cationically modified guar having weight average molecular weight in the range of from about 50 to 500 KDa, and (b) 1 to 99 wt. % of at least one cationically modified guar having weight average molecular weight in the range of from about 500 to 1500 KDa; (ii) 0.0 to 99 wt. % of a non-ionic guar; and (iii) 0.1 to 99 wt. % of at least one cosmetic ingredient.

In another aspect, the present application provides a method of protecting hair from the heat of hot flat ironing up to 450° F. comprising contacting the hair with a multi-modal hair care composition comprising: (i) 0.1 to 99 wt. % of at least one cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; (ii) 0.0 to 99 wt. % of a non-ionic guar; and (iii) 0.1 to 99 wt. % of at least one cosmetic ingredient.

### BRIEF DESCRIPTION OF DRAWINGS

In addition to the cited advantages and objects of the disclosure, one or more descriptions of the disclosure briefly summarized can be added by reference to certain embodiments thereof which are illustrated in the appended drawings. These drawings form part of the specification. However, it is to be noted that the appended drawings illustrate preferred embodiments of the disclosure and therefore are not limiting in their scope.

FIG. 1 illustrates tribological behavior of individual components as described in Tables 1, 3 & 4.

FIG. 2 illustrates tribological behavior of individual components (Table 1 & 2) compared to blends (Tables 7 & 8).

FIG. 3 illustrates on hair performance increase with blends over the individual components; examples based on optimizing weight ratios, molecular weight, and charge density.

FIG. 4 illustrates hair split end mending efficacy test using guar blend composition.

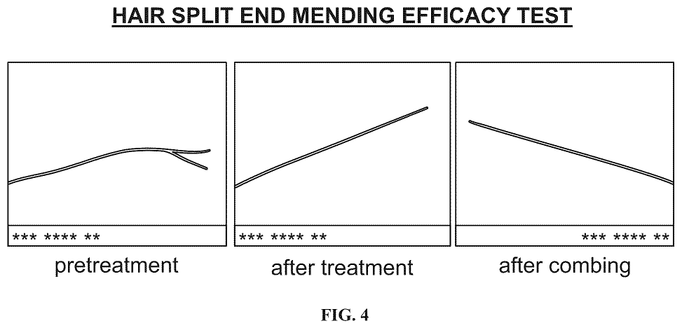

FIG. 5 illustrates stereomicroscopic results of split end mending efficacy after 1×leave-in treatment on damaged hair.

FIG. 6 illustrates stereomicroscopic results of split end mending efficacy after combing (mechanical durability) on damaged hair.

FIG. 7 illustrates stereomicroscopic results of split end mending efficacy after 1×leave-in treatment on damaged hair.

FIG. 8 illustrates stereomicroscopic results of split end mending efficacy after 1×leave-in treatment on damaged hair.

FIG. 9 illustrates thermal protection of hair from heat due to flat ironing.

FIG. 10 illustrates protection of hair from thermal & mechanical damage.

FIG. 11 illustrates thermal protection of hair from heat due to flat ironing.

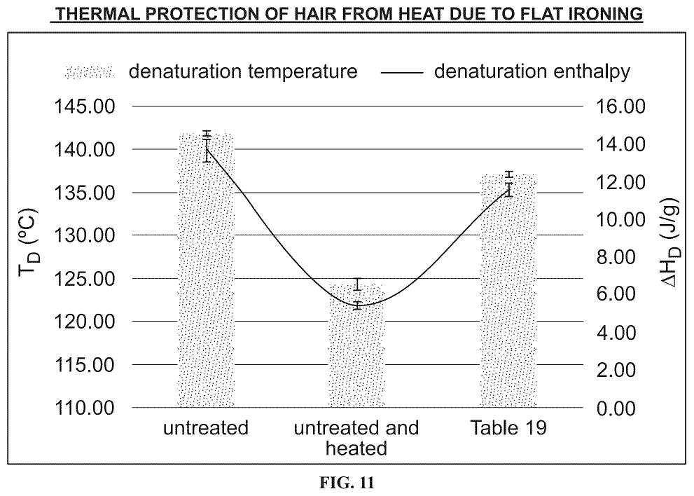

### DETAILED DESCRIPTION OF THE INVENTION

The technical solutions of the present disclosure will be described clearly and better understood in combination with specific embodiments below, but those skilled in the art will understand that embodiments described below are part of embodiments of the present disclosure but not all of them and are only used for illustration of the present disclosure and should not be considered as limiting the scope of the disclosure. If any specific condition or process are not indicated in the examples, it is to be understood that the conditions are conventionally used by the manufacturer and is commercially available.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by the context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

As used herein, the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the term “cationic polymers” refers to polymers displaying at least one primary amine, secondary amine or tertiary amine or quaternary ammonium group in their principal chain or branched chain or in the substituted form.

As used herein, the term “charge density” refers to the ratio of the number of charges on a polymer unit to the molecular weight of said polymer unit. Charge Density is further defined as the number of milliequivalents of charge per gram (meq/g) of polymer.

As used herein, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “cosmetically acceptable” refers to molecular entities regarded as safe, approved by regulatory body, listed in pharmacopoeia for use in topical contact with tissues (e.g., the skin) without undue toxicity, incompatibility, instability, irritation, allergic response, or the like. This term is not intended to limit the composition it describes as for use solely as a cosmetic (e.g., the composition may be used as a pharmaceutical).

As used herein, the term “degree of substitution (DS)” or “substituted degree” or “substitution degree” generally refers to number of cationic groups per anhydro sugar unit (cationic groups per galactomannan unit). Galactomannans are linear polysaccharides-(1,4)-linked D-mannose units substituted with D-galactose monomeric branches.

As used herein, the term “multi-modal” refers to multi-model refers to blends of cationic polysaccharides with different molecular weights and degree of substitution values. These blends encompass polysaccharides with multiple Molecular Weight and Degree of Substitution ranges.

As used herein, the term “polymer” refers to a compound comprising repeating structural units (monomers) connected by covalent chemical bonds. Polymers may be further derivatized, crosslinked, grafted or end capped. Non-limiting examples of polymers include copolymers, terpolymers, tetra polymers, quaternary polymers, and homologues. The term “copolymer” refers to a polymer consisting essentially of two or more different types of monomers polymerized to obtain said copolymer.

As used herein, the term “personal care composition” and “cosmetics” refer to compositions intended for use on or in human body such as skin, hair, oral including those to alter the color and appearance of skin and hair.

In one non-limiting embodiment, the present disclosure is directed to a multi-modal hair care composition, the composition comprising: (i) cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; and (ii) 1 to 99 wt. % of at least one cosmetic ingredient.

The present application employs cationic guar polymers in blends provided with different physical characteristics such as weight average molecular weights and degree of substitution. Such blends of cationic moieties with different molecular weights and degree of substitution values brings unique advantages such as customizable aesthetic and styling properties along with functional benefits to the hair care composition. Accordingly, the present application provides a multi-modal hair care composition, wherein the multi-modal implies cationic guars having different ranges of weight average molecular weights and degree of substitution. Accordingly, the multi-modal hair care composition is bi-modal, tri-modal, quadra-modal or penta-modal blend of cationic guar polymers.

Guar gum is a natural compound extracted from the seeds of guar bean (Cyamopsis tetragonoloba taub). Guar gum chemically comprises beta 1,4-glycosidic mannose units that statistically alternate between one alpha 1,6-bound galactose unit forming a side branch.

Guar polysaccharides can be substituted with derivatives encompassing cationic moieties which may be linked to the reactive functional group by a bivalent linking group, such as an alkylene or oxyalkylene group. Suitable cationic moieties include primary, secondary, or tertiary amino groups or quaternary ammonium, sulfonium, or phosphonium groups. Suitable quaternary ammonium groups are selected from trialkyl ammonium moieties, such as trimethylammonium moieties, triethylammonium moieties, or tributyl ammonium moieties, aryldialkyl ammonium moieties, such as benzyl dimethyl ammonium moieties. Cationic guars are polysaccharide derivatives, produced from the quaternization of guar gum. They have unique conditioning properties and are therefore widely used in shampoos, detergents, hair conditioners etc.

In another embodiment, the cationically modified guar blend is a combination of (a) cationic guar having weight average molecular weight in the range of from about 50 to about 500 KDa; and (b) cationically modified guar having weight average molecular weight in the range of from about 500 to about 1500 KDa.

In another embodiment, the cationically modified guar blend is a combination of (a) cationic guar having weight average molecular weight in the range of from about 200 to about 500 KDa; and (b) cationically modified guar having weight average molecular weight in the range of from about 800 to about 1200 KDa.

Accordingly, in a non-limiting embodiment of the present application, the weight average molecular weight of the cationic guar is in the range of from about 50 to about 100 KDa, from about 100 to about 150 KDa, from about 150 to about 200 KDa, from about 200 to about 250 KDa, from about 250 to about 300 KDa, from about 300 to about 350 KDa, from about 350 to about 400 KDa, from about 400 to about 450 KDa and from about 450 to about 500 KDa.

Accordingly, in a non-limiting embodiment of the present application, the weight average molecular weight of the cationic guar is in the range of from about 500 to about 600 KDa, from about 600 to about 700 KDa, from about 700 to about 800 KDa, from about 800 to about 900 KDa, from about 900 to about 1000 KDa, from about 1000 to about 1100 KDa, from about 1100 to about 1200 KDa, from about 1200 to about 1300 KDa, from about 1300 to about 1400 KDa, and from about 1400 to about 1500 KDa.

The disclosed weight average molecular weight range of cationically modified guar blend comprising (a) cationically modified guar having weight average molecular weight in the range of from about 50 to about 500 KDa; and (b) cationically modified guar having weight average molecular weight in the range of from about 500 to about 1500 KDa are present in weight ratio of 92:8, 80:20, 75:25, and 60:40.

In another embodiment, the cationicity of the polysaccharide derivative can also be expressed in terms of “degree of substitution” (DS). The degree of substitution of cationically modified guar blend comprising: (i) (a) cationically modified guar having weight average molecular weight in the range of from about 50 to about 500 KDa; and (b) cationically modified guar having weight average molecular weight in the range of from about 500 to about 1500 KDa; the blend (i) (a) or (i) (b) are present in ratio of 0.01 to 0.50, 0.01 to 0.10, 0.1 to 0.20, 0.2 to 0.30, and 0.3 to 0.4.

In another embodiment, the present application provides cationically modified guar blend comprising: (i) (a) cationically modified guar having weight average molecular weight in the range of from about 50 to about 500 KDa; and (b) cationically modified guar having weight average molecular weight in the range of from about 500 to about 1500 KDa; the blend of (i) (a) or (b) has a cationic degree of substitution in the range of 0.08 to 3.0.

Present application employs blends of cationic or cationically modified guars in preparing hair care compositions, wherein the cationically modified guar is selected from cationic hydroxyethyl guars, cationic hydroxypropyl guars, cassia hydroxypropyltrimonium chloride, and guar hydroxypropyltrimonium chloride. Hydroxypropyl Guar Hydroxypropyltrimonium Chloride (chemically, it is guar gum, 2-hydroxypropyl 2˜hydroxy-3-(trimethylammonio) propyl ether chloride); Guar Hydroxypropyl trimonium Chloride (chemically, it is guar gum, 2-hydroxy-3˜ (trimethylammonio) propyl ether, chloride); Caesalpinia Spinosa Hydroxypropyltrimonium Chloride (chemically, it is tara gum, 2-hydroxy-3-(trimethylammonio) propyl ether, chloride);

Locust Bean Hydroxypropyltrimonium Chloride; Trigonella Foenum-Graecum Hydroxypropyltrimonium Chloride.

In another non-limiting embodiment, the present composition provides a (i) blend of (a) guar hydroxypropyltrimonium chloride having molecular weight in the range of from about 50 to about 500 KDa, and (b) guar hydroxypropyltrimonium chloride having molecular weight in the range of from about 800 to about 1200 KDa; and degree of substitution (DS) in the range of 0.01 to 0.3, and (ii) blend of (a) guar hydroxypropyltrimonium chloride, and (b) cationic hydroxypropyl guar.

In another embodiment, the present hair care composition further comprises non-ionic guar. Non-ionic guars are selected from the group consisting of hydroxymethyl guar, hydroxyethyl guar, hydroxypropyl guar and hydroxybutyl guar.

In another embodiment, the present hair care composition can be used in a hair care formulation in the range of from about 0.01 wt. % to about 10.0 wt. %, and from about 0.01 to about 5.0 wt. % based on the total weight of the formulation.

In another non-limiting embodiment, the range may include from about 0.01 wt. % to about 1.0 wt. %; from about 1.0 wt. % to about 2.0 wt. %; from about 2.0 wt. % to about 3.0 wt. %; from about 3.0 wt. % to about 4.0 wt. %; and from about 4.0 wt. % to about 5.0 wt. %.

In another embodiment, the current hair care composition is used for hair styling, hair straightening, hair split end mending, hair cuticular smoothing, hair conditioning, protecting hair from thermal and mechanical damage, and protecting hair from thermal damage from heat styling tools up to 450° F.

The present hair care composition comprising cationically modified guar blends shows a pH in the range of from about 3 to about 13. Other non-limiting range of pH for the present include from about 3 to about 6; from about 6 to about 9; and from about 9 to about 13.

The hair care composition may contain at least one personal care additive including but not limited to acids, amino acids, antidandruff agents, anti-statics, antioxidants, anti-inflammatory agents, anti-microbial agents, acidifying agents, basifying agents, bodying agents, buffers, bleaches, cationic surfactants, cleansing agents, chelating agents, collagen, conditioning ingredients, compressed gases, dyes, de-ionized water, defoamers, emollients, emulsifiers, flexibility enhancers, fatty substances, fragrances, fillers, foam stabilizers, functional polymers, gel formers, humectants, α-hydroxycarboxylic acids, hydrophilic active agent, hair conditioning ingredients, hydrotropes, keratin hydrolyzates, light protection agents, lipophilic active agent, lipids, moisturizers, neutralizing agents, oxidizing agents, organosilicones, protein hydrolyzates, hydrotropes, perfumes, pigments, preservatives, propellants, proteins, pH modifier, polysaccharides, polyalklylene glycols, reducing agents, rheology modifiers, solvents, solubilizers, suspension agents, silicones, silicone-containing polymers, split modifiers, shine enhancers, softeners, sequestering agents, surfactants, styling polymers, thickeners, texturizing agents, tinting agents, tanning agents, UV-A or UV-B blocker/filters, UV protectants, viscosifiers, vitamins, water proofing agents, water softening agents, wetting agents, and blends thereof.

Non-limiting examples of polysaccharides include cassia, chitosan, chitin, dextrin, dextran, rosin, inulin, starch and starch derivatives, cellulose and cellulose derivatives, alginates, carrageenan, gellan, agar, pectin, arabinoxylan, pullulan and maltodextrin.

Non-limiting examples of antioxidants may be added to facilitate the enhanced shelf-life of the hair care composition of the present application. Exemplary antioxidants that can be used include vitamins such as vitamin E, vitamin E acetate, vitamin C, vitamin A, and vitamin D and derivatives thereof. Additional exemplary antioxidants include but are not limited to propyl, octyl and dodecyl esters of gallic acid, butylated hydroxyanisole (BHA), butylated hydroxytoluene (BHT), and nordihydroguaiaretic acid. In general, the required amount of antioxidant for the present composition is in the range of about 0.2 wt. % to about 2 wt. % and can be provided in an amount of about 0.5 wt. % to about 1.5 wt. %, based on the total weight of the composition.

Neutralizers or pH modifiers are employed to achieve the desired pH for the hair care composition. Non-limiting examples of neutralizing agents or pH modifiers include cosmetically acceptable bases selected from sodium and potassium hydroxide; organic bases such as methyl ethylamine (MEA), ammonia, amino-alcohols, lithium hydroxide, diethanolamine (DEA); triethanolamine (TEA), aminomethyl propanol, and mixtures thereof.

Non-limiting examples of neutralizing agents or pH modifiers include cosmetically acceptable acids selected from mineral acids, carboxylic acids, and polymeric acids. Specific examples for mineral acids are hydrochloric acid, nitric acid, phosphoric acid, and sulfuric acid. Examples for appropriate carboxylic acids are citric acid, glycolic acid, lactic acid, maleic acid, malic acid, succinic acid, glutaric acid, benzoic acid, malonic acid, salicylic acid, gluconic acid, and mixtures thereof. Examples for suitable polymeric acids include straight-chain poly(acrylic) acid and its copolymers (e.g., maleic-acrylic, sulfonic-acrylic, and styrene-acrylic copolymers), cross-linked polyacrylic acids having a molecular weight of less than about 2,50,000, poly(methacrylic) acid, and naturally occurring polymeric acids such as carageenic acid, carboxymethyl cellulose, and alginic acid. The neutralizing agent or pH modifier can be present in an amount of about 0.01 wt. % to about 8 wt. % of the total hair care composition.

Non-limiting examples of cationic surfactants include quaternary ammonium salts, such as di(C10-C24)-alkyl dimethylammonium chloride or bromide, preferably di(C12-C18)-alkyldimethyl ammonium chloride or bromide; (C10-C24)-alkyldimethylethyl ammonium chloride or bromide; (C10-C24)-alkyltrimethyl ammonium chloride or bromide, preferably cetyltrimethylammonium chloride or bromide and (C20-C22)-alkyltrimethyl ammonium chloride or bromide; (C10-C24)-alkyldimethylbenzyl ammonium chloride or bromide, preferably (C12-C18)-alkyldimethylbenzyl ammonium chloride; N-(C10-C18)-alkylpyridinium chloride or bromide, preferably N-(C12-C16)-alkylpyridinium chloride or bromide; N-(C10-C18)-alkylisoquinolinium chloride, bromide or monoalkyl sulfate; N-(C12-C18)-alkylpolyoyl amino formyl methyl pyridinium chloride; N-(C12-C18)-alkyl-N-methyl morpholinium chloride, bromide or monoalkyl sulfate; N-(C12-C18)-alkyl-N-ethyl morpholinium chloride, bromide or monoalkyl sulfate; (C16-C18)-alkylpentaoxyethyl ammonium chloride; diisobutyl phenoxyethoxy ethyldimethyl benzyl ammonium chloride; salts of N,N-diethylamino ethylstearylamide and oleylamide with hydrochloric acid, acetic acid, lactic acid, citric acid, phosphoric acid; N-acylaminoethyl-N,N-diethyl-N-methyl ammonium chloride, bromide or monoalkyl sulfate and N-acylaminoethyl-N,N-diethyl-N-benzyl ammonium chloride, bromide or monoalkyl sulfate, where acyl is preferably stearyl or oleyl.

Non-limiting examples of cationic surfactants are selected from the group consisting of cetrimonium chloride, cetrimonium bromide, dicetyldimonium chloride and palmitamidopropyl trimonium chloride.

Non-limiting examples of preservatives include phenoxyethanol, formaldehyde solution, parabens, penta-diol, sorbic acid, caprylyl glycol, raspberry ketone, benzoic acid, dehydroacetic acid, benzyl alcohol, propanediol, propylene carbonate, sodium benzoate, potassium sorbate, diazolidinyl urea, imidazolidinyl urea, iodopropynyl butylcarbamate, methylisothiazolinone, methylchloro isothiazolinone, phenyl propanol, 2-Bromo-2-Nitropropane1,3-Diol, Sodium Hydroxymethyl glycinate, 1,2-hexanediol, ethylhexylglycerin, Octenidine HCl, and chlorohexidine.

Non-limiting examples of pearlizing agents include glycol distearic esters, such as ethylene glycol distearate, but also fatty acid monoglycol esters or triethylene glycol distearate.

Non-limiting examples of coloring agents, colorants or dyes used herein include natural foods colors and dyes suitable for food, drug, and cosmetic applications. These colorants are also known as FD & C, and D&C dyes and lakes and are preferably water-soluble in nature.

In another embodiment, the present hair care composition can be formulated as an aerosol, a cream, an emulsion, a gel, a lotion, a mousse, a powder, a paste, a solution, spray, a shampoo, a vesicle dispersion, or a wax.

The present hair care composition is formulated as a mousse, a styling gel, a cream, or a hair wax, and wherein, (i) the compositions is in the form of is a bi-modal, tri-modal, quadra-modal or penta-modal; and (ii) the compositions comprise: (i) 0.1 to 99 wt. % of at least one cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; and (iii) 0.1 to 99 wt. % of at least one cosmetic ingredient.

In another embodiment, the present application provides a hair care composition for mending split ends comprising (i) 0.1 to 99 wt. % of at least one cationically modified guar blend comprising: (a) 1 to 99 wt % of at least one cationically modified guar having weight average molecular weight in the range of from about 50 to 500 KDa, and (b) 1 to 99 wt. % of at least one cationically modified guar having weight average molecular weight in the range of from about 500 to 1500 KDa; and (iii) 0.1 to 99 wt. % of at least one cosmetic ingredient.

The hair care composition is a leave-in composition. The hair care composition is a bi-modal, tri-modal, quadra-modal or penta-modal hair care composition.

In another embodiment, the present application provides a method for repairing split ends of hair comprising contacting hair with split ends with a multi-modal hair care composition comprising: (i) 0.1 to 99 wt. % of at least one cationically modified guar blend comprising: (a) 1 to 99 wt. % of at least one cationically modified guar having weight average molecular weight in the range of from about 50 to 500 KDa, and (b) 1 to 99 wt. % of at least one cationically modified guar having weight average molecular weight in the range of from about 500 to 1500 KDa; (ii) 0.0 to 99 wt. % of a non-ionic guar; and (iii) 0.1 to 99 wt. % of at least one cosmetic ingredient followed by rinsing the hair with water.

In another embodiment, the present application provides an aqueous hair mousse composition comprising: (i) 0.1 to 10 wt. % of a hair care composition comprising cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa, (ii) 1.0 to 60 wt. % of at least one solvent; (iii) 0.1 to 10 wt. % of at least one cosmetic functional additive; and (iv) 0.1 to 95 wt. % of water.

Selection of a propellant or blend of propellants may be used to achieve a particular spray pattern, control particle size, conform to government regulations, or for cost considerations. Propellants may be selected from the group consisting of hydrocarbons, hydrofluorocarbons, ethers, and combinations thereof. Non-limiting examples of propellants include pentane, n-butane, isobutane, propane, 1,1,1,2-tetrafluoroethane (134a). 1,1-difluoroethane (152a), dimethyl ether, compressed air, compressed nitrogen, compressed carbon dioxide, nitrous oxide, and mixtures thereof.

In one or more embodiments the total amount of propellant is from about 2 to about 97 wt. %, in other embodiments from about 4 to about 90 wt. %, and in other embodiments from about 5 to about 80 wt. %, based upon the total weight of the total composition.

Characterization of friction and wear properties of hair and scalp is essential to developing better shampoos and conditioners that leave hair untangled and looking natural while feeling smooth. Friction and wear experiments have been performed using a flat-on-flat tribometer under reciprocating motion to stimulate scalp-hair and hair-hair contacts at nominal conditions of temperature and humidity. The present hair care composition can deliver a wide range of tribological behavior as determined by 3-Balls on plate tribology attachment on a Rheometer and measurements made on Transpore TM surface and applied normal forces.

Details of equipment and set-up:

### Method:

Equipment ARES-G2 rheometer, TA instruments
3-Balls on plate tribology attachment on a Rheometer
Surfaces: Balls: steel with smooth surface; Strips: rough 3M Transpore tape

### Settings

Load force-5N, T=25° C.; Angular Velocity: ramp

The present composition exhibits improved humidity resistance, stiffness, durability of hold, low slip, foam enhancement and curl maintenance. The composition exhibits a high humidity curl retention of about 80% to about 100% over a 24-hour period at 90% humidity and at 80° F.

As the present composition comprises cationic modified guars which are nature derived and are biodegradable.

The multimodal composition enables improved curl maintenance, variations of hold and stiffness, while delivering improvement in the delivery aesthetics such cushion in the and slip through the hair.

In another embodiment, the present application provides a method for thermal and mechanical protection from brush and blow drying, comprising contacting the hair with a multi-modal hair care composition comprising: (i) 0.1 to 99 wt. % of at least one cationically modified guar blend comprising: (a) 1 to 99 wt. % of at least one cationically modified guar having weight average molecular weight in the range of from about 50 to 500 KDa, and (b) 1 to 99 wt. % of at least one cationically modified guar having weight average molecular weight in the range of from about 500 to 1500 KDa; (ii) 0.0 to 99 wt. % of a non-ionic guar; and (iii) 0.1 to 99 wt. % of at least one cosmetic ingredient.

In another embodiment, the present application provides a method of protecting hair from heat of hot flat ironing up to 450° F. comprising contacting the hair with a multi-modal hair care composition comprising: (i) 0.1 to 99 wt. % of at least one cationically modified guar blend comprising: (a) 1 to 99 wt. % of at least one cationically modified guar having weight average molecular weight in the range of from about 50 to 500 KDa, and (b) 1 to 99 wt. % of at least one cationically modified guar having weight average molecular weight in the range of from about 500 to 1500 KDa; (ii) 0.0 to 99 wt. % of a non-ionic guar; and (iii) 0.1 to 99 wt. % of at least one cosmetic ingredient.

Further, certain aspects of the present application are illustrated in detail by way of the following examples. The examples are given herein for illustration of the application and are not intended to be limiting thereof.

### EXAMPLES

The hair care composition prepared using cationically modified guar polymer blends were effectively subjected to tests to compare and analyze the performance of present polymer blends with that of commercially available synthetic polymers. The present composition is compared against commercial synthetic polymers polyquaternium-11 (PQ-11) & polyquaternium-4 (PQ-4).

### Commercial Controls

### Example 1: A Mousse Composition (Comparative Example)

A control sample was prepared using the following ingredients.

TABLE 1
Commercial Control

| Ingredients | Amount (%) |
|---|---|
| Water | 90.79 |
| Polyquaternium-11 | 2.00 |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Example 2: A Mousse Composition (Comparative Example)

A control sample was prepared using the following ingredients.

TABLE 2
Commercial Control

| Ingredients | Amount (%) |
|---|---|
| Water | 90.79 |
| Polyquaternium-4 | 2.00 |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Individual Guars

### Example 3: A Mousse Composition

A mousse was prepared using the following ingredients.

TABLE 3
A Mousse

| Ingredients | Amount (%) |
|---|---|
| Water | 90.79 |
| Guar hydroxypropyltrimonium chloride | 2.00 |
| [Mol. Wt. 200-500 KDa; DS - 0.14-0.16] | |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Example 4: A Mousse Composition

A mousse was prepared using the following ingredients.

TABLE 4
A Mousse

| Ingredients | Amount (%) |
|---|---|
| Water | 90.79 |
| Guar hydroxypropyltrimonium chloride | 2.0 |
| [Mol. Wt. 800-1200 KDa; DS: 0.13-0.16] | |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Example 5: A Mousse Composition

A mousse was prepared using the following ingredients.

TABLE 5
A Mousse

| Ingredients | Amount (%) |
|---|---|
| Water | 90.79 |
| Guar hydroxypropyltrimonium chloride | 2.00 |
| [Mol. Wt. 800-1200 KDa; DS: 0.17-0.20] | |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Example 6: A Mousse Composition

A mousse was prepared using the following ingredients.

TABLE 6
A Mousse

| Ingredients | Amount (%) |
|---|---|
| Water | 90.79 |
| Guar hydroxypropyltrimonium chloride | 2.0 |
| [Mol. 800-1200 KDa; DS - 0.24-0.30] | |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Bi-modal Guar Blends

### Example 7: A Mousse Composition

Mousse Composition prepared using blends of guar hydroxypropyl trimonium chloride having different ranges of molecular weight and degree of substitution.

TABLE 7
A Mousse

| Ingredients | Amount (%) |
|---|---|
| Water | 90.79 |
| Guar hydroxypropyltrimonium chloride | 1.50 |
| [Mol. Wt. - 200-500 KDa; DS - 0.12-0.14] | |
| Guar hydroxypropyltrimonium chloride | 0.50 |
| [Mol. Wt. 800-1200 KDa; DS - 0.13-0.16] | |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Example 8: A Mousse Composition

Mousse Composition was prepared using blends of guar hydroxypropyl trimonium chloride having different ranges of molecular weight and degree of substitution.

TABLE 8
A Mousse

| Ingredients | Amount %) |
|---|---|
| Water | 90.79 |
| Guar hydroxypropyltrimonium chloride | 1.50 |
| [Mol. Wt. - 200-500 KDa; DS - 0.12-0.14] | |
| Guar hydroxypropyltrimonium chloride | 0.50 |
| [Mol. Wt. 800-1200 KDa; DS - 0.17-0.20] | |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Example 9: A mousse composition

Mousse Composition was prepared using guar hydroxypropyl trimonium chloride and hydroxypropyl guar.

TABLE 9
A Mousse

| Ingredients | Amount (%) |
|---|---|
| Water | 90.79 |
| Guar hydroxypropyltrimonium chloride | 1.50 |
| [Mol. Wt. - 200-500 KDa; DS - 0.12-0.14] | |
| Hydroxypropyl Guar (non-ionic) | 0.50 |
| [DS - 0.12-0.14] | |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Bi-Modal Quar Blends

### Example

1% polymer was prepared using blend of guars with different molecular weights and the composition details are provided below.

TABLE 10
Guar Blends

| | Ex. 10 | Ex. 11 | Ex. 12 | Ex. 13 | Ex. 14 | Ex. 15 |
|---|---|---|---|---|---|---|
| Ingredients | wt. % | wt. % | wt. % | wt. % | wt. % | wt. % |
| Guar hydroxypropyltrimonium chloride | 1.25 | 1.25 | 1.25 | — | — | — |
| [Mol. wt. 50-100 KDa; DS: 0.17-0.20] | | | | | | |
| Guar hydroxypropyltrimonium chloride | 0.75 | 0.75 | 0.75 | 0.75 | 0.75 | 0.75 |
| [Mol. wt. 200-500 KDa; DS: 0.14-0.16] | | | | | | |
| Cationic cassia | 0.13 | — | — | — | 0.13 | 0.13 |
| [Mol. wt. 600-900 KDa; DS: 0.25-0.30] | | | | | | |
| Guar hydroxypropyltrimonium chloride | — | 0.13 | — | — | 0.13 | — |
| [Mol. wt. 800-1200 KDa; DS: 0.13-0.16] | | | | | | |
| Guar hydroxypropyltrimonium chloride | — | — | — | — | — | 0.13 |
| [Mol. wt. 800-1200 KDa; DS: 0.17-0.20] | | | | | | |
| Guar hydroxypropyltrimonium chloride | — | — | 0.13 | — | — | — |
| [Mol. wt. 800-1200 KDa; DS: 0.24-0.30] | | | | | | |
| Hydroxypropyl Guar Hydroxypropyltrimonium | — | — | — | 0.13 | — | — |
| Chloride | | | | | | |
| [Mol. wt. 800-1200 KDa; DS: 0.08-0.10] | | | | | | |
| NaOH (10% soln | — | — | — | — | qs | — |
| lactic acid | 0.25 | 0.25 | — | 0.25 | 0.25 | 0.25 |
| optiphen | 0.750 | 0.750 | 0.750 | 0.750 | 0.750 | 0.750 |
| Water | 96.88 | 96.88 | 97.13 | 96.88 | 98.05 | 98.00 |
| Total | 100.00 | 100.00 | 100.00 | 100.00 | 100.00 | 100.00 |

### Example 16

Around 2% total polymer was prepared using a blend of guars with different molecular weights and the composition details are provided below.

TABLE 11
Guar Blends

| | Blend | | | | | |
|---|---|---|---|---|---|---|
| | A | B | C | D | E | F |
| Ingredients | wt. % | wt. % | wt. % | wt. % | wt. % | wt. % |
| Guar hydroxypropyltrimoniumchloride | — | — | — | — | — | — |
| [Mol. wt. 200-500 KDa; DS: 0.14-0.16] | | | | | | |
| Guar hydroxypropyltrimoniumchloride | — | — | — | — | 1.00 | 0.80 |
| [Mol. wt. 800-1200 KDa; DS: 0.13-0.16] | | | | | | |
| Guar hydroxypropyltrimoniumchloride | 1.00 | 0.80 | 0.40 | 0.20 | — | — |
| [Mol. wt. 800-1200 KDa; DS: 0.17-0.20] | | | | | | |
| Hydroxypropyl Guar | | | | | | |
| Hydroxypropyltrimonium Chloride | 1.00 | 1.20 | 1.60 | 1.80 | 1.00 | 1.20 |
| [Mol. Wt.800-1200 KDa; DS: 0.08-0.10] | | | | | | |
| Citric Acid | 0.06 | 0.06 | 0.06 | 0.06 | 0.08 | 0.08 |
| Liquid Germall Plus | 0.20 | 0.20 | 0.20 | 0.20 | 0.20 | 0.20 |
| Water | 97.74 | 97.74 | 97.74 | 97.74 | 97.72 | 97.72 |
| Total | 100.00 | 100.00 | 100.00 | 100.00 | 100.00 | 100.00 |
| | Blend | | | | | |
| | G | H | I | J | K | L |
| Ingredients | wt. % | wt. % | wt. % | wt. % | wt. % | wt. % |
| Guar hydroxypropyltrimonium chloride | — | — | 1.00 | 0.80 | 0.40 | 0.20 |
| [Mol. wt. 200-500 KDa; DS: 0.14-0.16] | | | | | | |
| Guar hydroxypropyltrimonium chloride | 0.4 | 0.2 | — | — | — | — |
| [Mol. wt. 800-1200 KDa; DS: 0.13-0.16] | | | | | | |
| Guar hydroxypropyltrimonium chloride | | | | | | |
| [Mol. wt. 800-1200 KDa; DS: 0.17-0.20] | — | — | — | — | — | — |
| Hydroxypropyl Guar | | | | | | |
| Hydroxypropyltrimonium Chloride | 1.60 | 1.80 | 1.00 | 1.20 | 1.60 | 1.80 |
| [Mol. Wt. 800-1200 KDa; DS: 0.08-0.10] | | | | | | |
| Citric Acid | 0.08 | 0.08 | 0.07 | 0.07 | 0.07 | 0.07 |
| Liquid Germall Plus | 0.20 | 0.20 | 0.20 | 0.20 | 0.20 | 0.20 |
| Water | 97.72 | 97.72 | 97.73 | 97.73 | 97.73 | 97.73 |
| Total | 100.0 | 100.0 | 100.0 | 100.0 | 100.0 | 100.0 |

### Example 17

Around 1% total polymer was prepared using a blend of guars with different molecular weights and the composition details are provided below.

TABLE 12
Guar Blends

| | Blend | | | | | | | |
|---|---|---|---|---|---|---|---|---|
| | M | N | O | P | Q | R | S | T |
| Ingredients | wt. % | wt. % | wt. % | wt. % | wt. % | wt. % | wt. % | wt. % |
| Guar hydroxypropyltrimonium chloride | — | — | — | — | 0.50 | 0.40 | 0.20 | 0.10 |
| [Mol. wt. 200-500 KDa; DS: 0.14-0.16] | | | | | | | | |
| Guar hydroxypropyltrimonium chloride | 0.50 | 0.40 | 0.20 | 0.10 | — | — | — | — |
| [Mol. wt. 800-1200 KDa; DS: 0.13-0.16] | | | | | | | | |
| Guar hydroxypropyltrimonium chloride | — | — | — | — | — | — | — | — |
| [Mol. wt. 800-1200 KDa; DS: 0.17-0.20] | | | | | | | | |
| Hydroxypropyl Guar | | | | | | | | |
| Hydroxypropyltrimonium Chloride | 0.50 | 0.60 | 0.80 | 0.90 | 0.50 | 0.60 | 0.80 | 0.90 |
| [Mol. Wt. 800-1200 KDa; DS: 0.08-0.10] | | | | | | | | |
| Citric Acid | 0.08 | 0.08 | 0.08 | 0.08 | 0.07 | 0.07 | 0.07 | 0.07 |
| Liquid Germall Plus | 0.20 | 0.20 | 0.20 | 0.20 | 0.20 | 0.20 | 0.20 | 0.20 |
| Water | 98.72 | 98.72 | 98.72 | 98.72 | 98.73 | 98.73 | 98.73 | 98.73 |
| Total | 100.00 | 100.0 | 100.00 | 100.00 | 100.00 | 100.00 | 100.00 | 100.00 |

### Tri-modal Guar Blends

### Example 18

Mousse Composition was prepared using blends of guar hydroxypropyl trimonium chloride having different ranges of molecular weight and degree of substitution.

TABLE 13
A Mousse

| Ingredients | Amount (%) |
|---|---|
| Water | 90.79 |
| Guar hydroxypropyltrimonium chloride | 1.50 |
| [Mol. Wt. 200-500 KDa; DS: 0.12-0.14] | |
| Guar hydroxypropyltrimonium chloride | 0.25 |
| [Mol. Wt. 800-1200 KDa; DS: 0.13-0.16] | |
| Guar hydroxypropyltrimonium chloride | 0.25 |
| [Mol. Wt. 800-1200 KDa; DS: 0.17-0.20] | |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Example 19

Mousse composition was prepared using blends of guar hydroxypropyl trimonium chloride having different ranges of molecular weight and degree of substitution.

TABLE 14
A Mousse

| Ingredients | Amount (%) |
|---|---|
| Water | 90.79 |
| Guar hydroxypropyltrimonium chloride | 1.50 |
| [Mol. Wt. 200-500 KDa; DS: 0.14-0.16] | |
| Guar hydroxypropyltrimonium chloride | 0.15 |
| [Mol. Wt. 800-1200 KDa; DS: 0.13-0.16] | |
| Guar hydroxypropyltrimonium chloride | 0.35 |
| [Mol. Wt. 800-1200 KDa; DS: 0.17-0.20] | |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Example 20

Mousse Composition was prepared using blends of guar hydroxypropyl trimonium chloride having different ranges of molecular weight and degree of substitution.

TABLE 15
A Mousse

| Ingredients | Amount (%) |
|---|---|
| Water | 90.79 |
| Guar hydroxypropyltrimonium chloride | 1.60 |
| [Mol. Wt. 200-500 KDa; DS: 0.12-0.14 gm/cm³] | |
| Guar hydroxypropyltrimonium chloride | 0.15 |
| [Mol. Wt. 800-1200 KDa; DS: 0.13-0.16 gm/cm³] | |
| Guar hydroxypropyltrimonium chloride | 0.25 |
| [Mol. Wt. 800-1200 KDa; DS: 0.17-0.201 gm/cm³] | |
| Lactic acid | 0.06 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Example 21

Mousse Composition was prepared using blends of guar hydroxypropyl trimonium chloride having different ranges of molecular weight and degree of substitution.

TABLE 16
A Mousse

| Ingredients | Amount (%) |
|---|---|
| Water | 90.79 |
| Guar hydroxypropyltrimonium chloride | 1.60 |
| [Mol. Wt. 200-500 KDa; DS: 0.14-0.16] | |
| Guar hydroxypropyltrimonium chloride | 0.25 |
| [Mol. Wt. 800-200 KDa; DS: 0.17-0.20] | 0.15 |
| Guar hydroxypropyltrimonium chloride | |
| [Mol. Wt. 800-200 KDa; DS: 0.24-0.30] | |
| Lactic acid | 0.06 |
| PPG-2 Hydroxyethyl Cocamide | 0.15 |
| Cctrimonium chloride (29%) | 0.25 |
| phenoxy ethanol and caprylyl glycol | 0.75 |
| Isobutane | 6.00 |
| Total | 100.00 |

### Example 22

Mousse Composition was prepared using blends of guar hydroxypropyl trimonium chloride having different ranges of molecular weight and degree of substitution.

TABLE 17
A Mousse

| Ingredients | Amount (%) |
|---|---|
| Water | 90.27 |
| Guar hydroxypropyltrimonium chloride | 2.00 |
| [Mol. Wt. 200-500 KDa; DS: 0.14-0.16] | |
| Guar hydroxypropyltrimonium chloride | 0.19 |
| [Mol. Wt. 800-1200 KDa; DS: 0.13-0.16] | |
| Guar hydroxypropyltrimonium chloride | 0.31 |
| [Mol. Wt. 800-1200 KDa; DS: 0.24-0.30] | |
| Lactic acid | 0.08 |
| PPG-2 Hydroxyethyl Cocamide | 0.15 |
| Cetrimonium chloride (29%) | 0.25 |
| phenoxy ethanol and caprylyl glycol | 0.75 |
| Isobutane | 6.00 |
| Total | 100.00 |

### Example

Trimodal Guar blends using cationic cassia, guar hydroxypropyl trimonium chloride and Hydroxypropyl Guar Hydroxypropyltrimonium Chloride with three different molecular weight ranges as tabulated below.

TABLE 18
Guar Blends Compositions

| | Ex. 23 | Ex. 24 | Ex. 25 | Ex. 26 | Ex. 27 | Ex. 28 |
|---|---|---|---|---|---|---|
| Ingredients | wt. % | wt. % | wt. % | wt. % | wt. % | wt. % |
| Guar hydroxypropyltrimonium chloride | 1.25 | 1.25 | 1.25 | 1.25 | — | — |
| [Mol. wt. 50-100 KDa; DS: 0.17-0.20] | | | | | | |
| Guar hydroxypropyltrimonium chloride | 0.75 | 0.75 | 0.75 | 0.75 | 0.75 | 0.75 |
| [Mol. wt. 200 -500 KDa; DS: 0.14-0.16] | | | | | | |
| Cationic cassia | 0.13 | — | — | — | 0.13 | 0.13 |
| [Mol. wt. 600-900 KDa; DS: 0.25-0.30] | | | | | | |
| Guar hydroxypropyltrimonium chloride | — | 0.13 | — | — | 0.13 | — |
| [Mol. wt. 800-1200 KDa; DS: 0.13-0.16] | | | | | | |
| Guar hydroxypropyltrimonium chloride | — | — | — | — | — | 0.13 |
| [Mol. wt. 800-1200 KDa; DS: 0.17-0.20] | | | | | | |
| Guar hydroxypropyltrimonium chloride | — | — | 0.13 | — | — | — |
| [Mol. wt. 800-1200 KDa; DS: 0.24-0.30] | | | | | | |
| Hydroxypropyl Guar | — | — | — | 0.13 | — | — |
| Hydroxypropyltrimonium Chloride | | | | | | |
| [Mol. wt. 800-1200 KDa; DS: 0.08-0.10] | | | | | | |
| NaOH (10% soln | — | — | — | — | qs | — |
| Lactic acid | 0.25 | 0.25 | | 0.25 | 0.20 | 0.25 |
| Optiphen | 0.75 | 0.75 | 0.75 | 0.75 | 0.75 | 0.75 |
| Water | 96.88 | 96.88 | 97.13 | 96.88 | 98.05 | 98.00 |
| Total | 100.0 | 100.00 | 100.00 | 100.00 | 100.00 | 100.00 |

### Example

Another set of tri-modal Guar blends using cationic cassia, guar hydroxypropyl trimonium chloride and Hydroxypropyl Guar Hydroxypropyltrimonium Chloride with three different molecular weight ranges as tabulated below.

TABLE 19
Guar Blends Composition

| | Ex. 29 | Ex. 30 | Ex. 31 | Ex. 32 | Ex. 33 | Ex. 34 | Ex. 35 |
|---|---|---|---|---|---|---|---|
| Ingredients | wt. % | wt. % | wt. % | wt. % | wt. % | wt. % | wt. % |
| Guar hydroxypropyltrimonium chloride | 0.75 | 0.75 | 0.75 | 0.75 | 0.75 | 0.75 | 0.75 |
| [Mol. wt. 200-500 KDa; DS: 0.14-0.16] | | | | | | | |
| Cationic cassia | 0.13 | 0.13 | — | — | — | — | — |
| [Mol. wt. 600-900 KDa; DS: 0.25-0.30] | | | | | | | |
| Guar hydroxypropyltrimonium chloride | — | — | 0.13 | 0.13 | 0.13 | — | — |
| [Mol. wt. 800-1200 KDa; DS: 0.13-0.16] | | | | | | | |
| Guar hydroxypropyltrimonium chloride | — | — | 0.13 | — | — | 0.13 | 0.13 |
| [Mol. wt. 800-1200 KDa; DS: 0.17-0.20] | | | | | | | |
| Guar hydroxypropyltrimonium chloride | 0.13 | — | — | 0.13 | — | 0.13 | — |
| [Mol. wt. 800-1200 KDa; DS: 0.24-0.30] | | | | | | | |
| Hydroxypropyl Guar | — | 0.13 | — | — | 0.13 | — | 0.13 |
| Hydroxypropyltrimonium Chloride | | | | | | | |
| [Mol. wt. 800-1200 KDa; DS: 0.08-0.10] | | | | | | | |
| NaOH (10% soln | qs | — | — | qs | — | qs | — |
| lactic acid | — | 0.30 | 0.25 | — | 0.30 | — | 0.25 |
| optiphen | 0.75 | 0.75 | 0.75 | 0.75 | 0.75 | 0.75 | 0.75 |
| Water | 98.25 | 97.95 | 98.00 | 98.25 | 97.95 | 98.25 | 98.00 |
| Total | 100.00 | 100.00 | 100.00 | 100.00 | 100.00 | 100.00 | 100.00 |

### Quadra-Modal Guar Blends

### Example

Quadra-modal Guar blends using cationic cassia, guar hydroxypropyl trimonium chloride and Hydroxypropyl Guar Hydroxypropyltrimonium Chloride with three different molecular weight ranges as tabulated below.

TABLE 20
Guar Blends

| | Ex.36 | Ex.37 | Ex.38 | Ex.39 | Ex.40 |
|---|---|---|---|---|---|
| Ingredients | wt. % | wt. % | wt. % | wt. % | wt. % |
| Guar hydroxypropyltrimonium chloride | 1.00 | 1.00 | 1.00 | 1.00 | — |
| [Mol. wt. 50-100 KDa; DS: 0.17-0.20] | | | | | |
| Guar hydroxypropyltrimonium chloride | 0.70 | 0.70 | 0.70 | 0.70 | 0.70 |
| [Mol. wt. 200-500 KDa; DS: 0.14-0.16] | | | | | |
| Cationic cassia | 0.10 | 0.10 | 0.10 | 0.10 | 0.10 |
| [Mol. wt. 600-900 KDa; DS: 0.25-0.30] | | | | | |
| Guar hydroxypropyltrimonium chloride | 0.10 | — | — | — | 0.10 |
| [Mol. wt. 800-1200 KDa; DS: 0.13-0.16] | | | | | |
| Guar hydroxypropyltrimonium chloride | — | 0.10 | — | — | 0.10 |
| [Mol. wt. 800-1200 KDa; DS: 0.17-0.20] | | | | | |
| Guar hydroxypropyltrimonium chloride | — | — | 0.10 | — | — |
| [Mol. wt. 800-1200 KDa; DS: 0.24-0.30] | | | | | |
| Hydroxypropyl Guar | — | — | — | 0.10 | — |
| Hydroxypropyltrimonium Chloride | | | | | |
| [Mol. wt. 800-1200 KDa; DS: 0.08-0.10] | | | | | |
| NaOH (10% soln) | — | — | qs | — | — |
| lactic acid | 0.30 | 0.25 | — | 0.25 | 0.25 |
| optiphen | 0.75 | 0.75 | 0.75 | 0.75 | 0.75 |
| Water | 97.05 | 97.10 | 97.35 | 97.10 | 98.00 |
| Total | 100.00 | 100.00 | 100.00 | 100.00 | 100.00 |

### Example

Quadra-modal Guar blends using cationic cassia, guar hydroxypropyl trimonium chloride and Hydroxypropyl Guar Hydroxypropyltrimonium Chloride with three different molecular weight ranges as tabulated below.

TABLE 21
Guar Blends

| | Ex.41 | Ex.42 | Ex.43 | Ex.44 | Ex.45 |
|---|---|---|---|---|---|
| Ingredients | Wt. % | Wt. % | Wt. % | Wt. % | Wt. % |
| Guar hydroxypropyltrimonium chloride | 0.70 | 0.70 | 0.70 | 0.70 | 0.70 |
| [Mol. wt. 200-500 KDa; DS: 0.14-0.16] | | | | | |
| Cationic cassia | 0.10 | 0.10 | — | — | — |
| [Mol. wt. 600-900 KDa; DS: 0.25-0.30] | | | | | |
| Guar hydroxypropyltrimonium chloride | 0.10 | 0.10 | 0.10 | 0.10 | — |
| [Mol. wt. 800-1200 KDa; DS: 0.13-0.16] | | | | | |
| Guar hydroxypropyltrimonium chloride | — | — | 0.10 | — | 0.10 |
| [Mol. wt. 800-1200 KDa; DS: 0.17-0.20] | | | | | |
| Guar hydroxypropyltrimonium chloride | 0.10 | — | 0.10 | 0.10 | 0.10 |
| [Mol. wt. 800-1200 KDa; DS: 0.24-0.30] | | | | | |
| Hydroxypropyl Guar Hydroxypropyl | — | 0.10 | — | 0.10 | 0.10 |
| trimonium Chloride | | | | | |
| [Mol. wt. 800-1200 KDa; DS: 0.08-0.10] | | | | | |
| NaOH (10% soln) | qs | — | qs | qs | qs |
| lactic acid | — | 0.30 | — | — | — |
| Optiphen | 0.75 | 0.75 | 0.75 | 0.75 | 0.75 |
| Water | 98.25 | 97.95 | 98.25 | 98.25 | 98.25 |
| Total | 100.00 | 100.00 | 100.00 | 100.00 | 100.00 |

### Penta-Penta Modal Guar Blends

### Example

Guar blends using cationic cassia, guar hydroxypropyl trimonium chloride and Hydroxypropyl Guar Hydroxypropyltrimonium Chloride with different molecular weight ranges as tabulated below:

TABLE 22
Guar Blends Compositions

| | Ex. 46 | Ex. 47 | Ex. 48 | Ex. 49 | Ex. 50 | Ex. 51 |
|---|---|---|---|---|---|---|
| Ingredients | wt. % | wt. % | wt. % | wt. % | wt. % | wt. % |
| Guar hydroxypropyltrimonium chloride | 1.000 | 1.000 | 1.000 | — | — | — |
| [Mol. wt. 50-100 KDa; DS - 0.17-0.20] | | | | | | |
| Guar hydroxypropyltrimonium chloride | 0.600 | 0.600 | 0.600 | 0.600 | 0.600 | 0.600 |
| [Mol. wt. 200-500 KDa; DS - 0.14-0.16] | | | | | | |
| Cationic cassia | 0.100 | 0.100 | 0.100 | 0.100 | 0.100 | — |
| [Mol. wt. 600-900 KDa; DS - 0.25-0.30] | | | | | | |
| Guar hydroxypropyltrimonium chloride | 0.100 | 0.100 | 0.100 | 0.100 | — | 0.100 |
| [Mol. wt. 800-1200 KDa; DS - 0.13-0.16] | | | | | | |
| Guar hydroxypropyltrimonium chloride | 0.100 | — | — | 0.100 | 0.100 | 0.100 |
| [Mol. wt. 800-1200 KDa; DS - 0.17-0.20] | | | | | | |
| Guar hydroxypropyltrimonium chloride | — | 0.100 | — | 0.100 | 0.100 | 0.100 |
| [Mol. wt. 800-1200 KDa DS - 0.24-0.30] | | | | | | |
| Hydroxypropyl Guar | — | — | 0.100 | — | 0.100 | 0.100 |
| Hydroxypropyltrimonium Chloride | | | | | | |
| [Mol. wt. 800-1200 KDa; DS - 0.08-0.10] | | | | | | |
| lactic acid | 0.25 g | — | 0.20 g | — | — | — |
| Optiphen | 0.75 | 0.75 | 0.75 | 0.75 | 0.75 | 0.75 |
| Water | 97.35 | 97.35 | 97.35 | 98.25 | 98.25 | 98.25 |
| Total | 100.00 | 100.00 | 100.00 | 100.00 | 100.00 | 100.00 |

### Example 52: High Humidity Curl Retention Test Results

Samples from Tables 1, 7, 8 & 9 were subjected to high humidity curl retention (HHCR) tests (90% relative humidity, 80° F. over 48 hrs.) and results are tabulated below.

### Method:

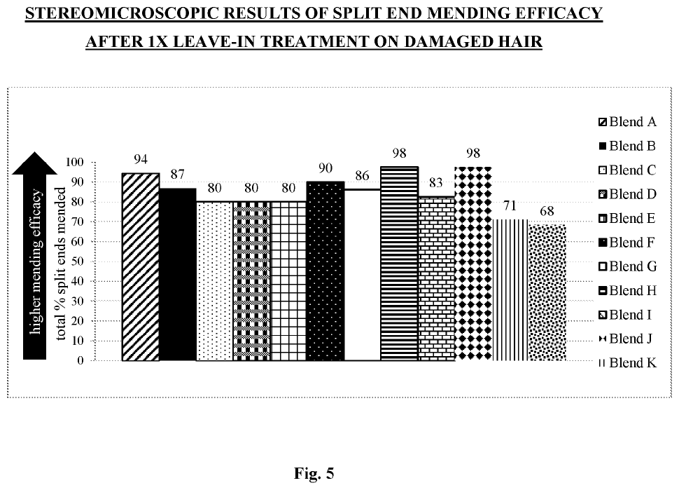

Humidity resistance-0.30 g mousse was applied to clean damp tress (International Hair Importers; Dark brown European, 2.0 g, 1.0″ wide, 6.5″ length) and rolled diagonally on 0.625″ roller, dried under diffuse heat and equilibrated to 40 RH, 25° C. Roller was removed, and the tress was hung on horizontally lined board marked in 0.25″ delineations and placed in high humidity environment (90RH, 27° C.). Initial reading for tress length was measured at the bottom of the curl. Additional readings were taken over 48 hrs and are shown in Table 21. Humidity resistance was calculated by the following equation: (FIG. 5)

$$( L o - Lt / Lo - Li ) \times 100 at time t$$

where: Lo=length of fully extended hair.
Li=length of hair before exposure (initial)
Lt=length of hair after exposure time (t)

TABLE 23
HHCR Test Results

| | Average % Retention | | | | | |
|---|---|---|---|---|---|---|
| | Time (minutes) | | | | | |
| Sample | 0 | 930 | 1140 | 1320 | 2280 | 2880 |
| Sample A | 100.00 | 15.17 | 10.86 | 10.06 | 6.86 | 5.99 |
| St dev | | 3.47 | 6.18 | 5.57 | 5.89 | 3.95 |
| Sample B | 100.00 | 96.69 | 96.69 | 96.69 | 96.69 | 96.69 |
| St. dev | | 1.87 | 1.87 | 1.87 | 1.87 | 1.87 |
| Sample C | 100.00 | 98.40 | 98.40 | 98.40 | 98.40 | 98.40 |
| St. dev | | 2.19 | 2.19 | 2.19 | 2.19 | 2.19 |
| Sample D | 100.00 | 98.36 | 98.36 | 98.36 | 98.36 | 98.36 |
| St. dev | | 2.17 | 2.17 | 2.17 | 2.17 | 2.17 |
| Sample E | 100.00 | 99.13 | 99.13 | 99.13 | 99.13 | 99.13 |
| St. dev | | 1.94 | 1.94 | 1.94 | 1.94 | 1.94 |
| Sample F | 100.00 | 100.00 | 100.00 | 100.00 | 100.00 | 100.00 |
| St. dev | | 0.00 | 0.00 | 0.00 | 0.00 | 0.00 |

### Test Results Observations:

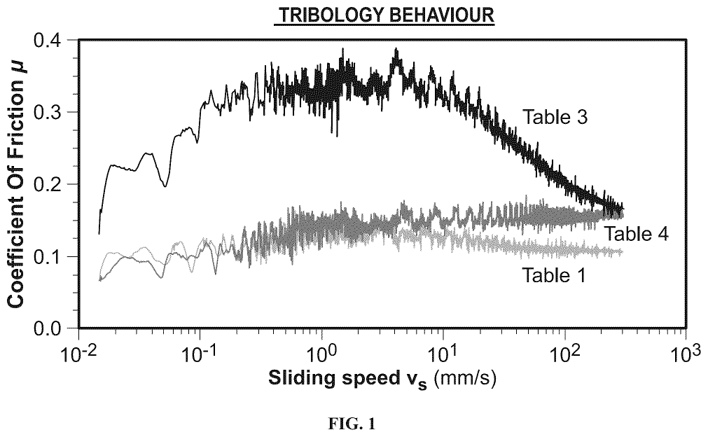

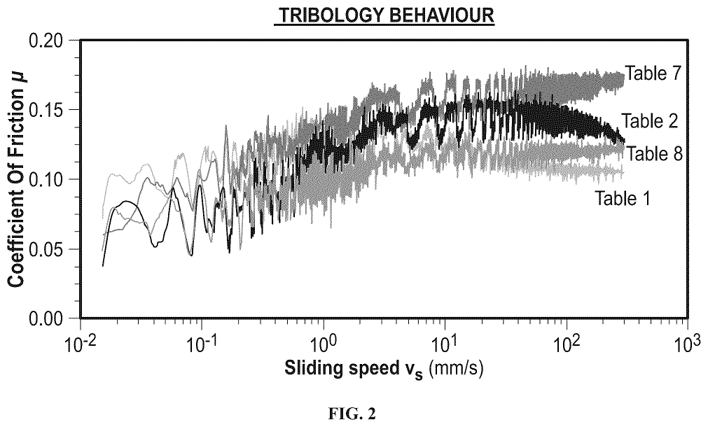

Examples provided above disclose individual samples from Table 1, 2, 3 and 4; and guar blends from tables 7 and 8. FIG. 1 discloses tribology behavior of individual Table 1, 3 and 4. FIG. 2 discloses tribology behavior of individual samples (Tables 1 and 2) compared to guar blend samples (Tables 7 and 8).

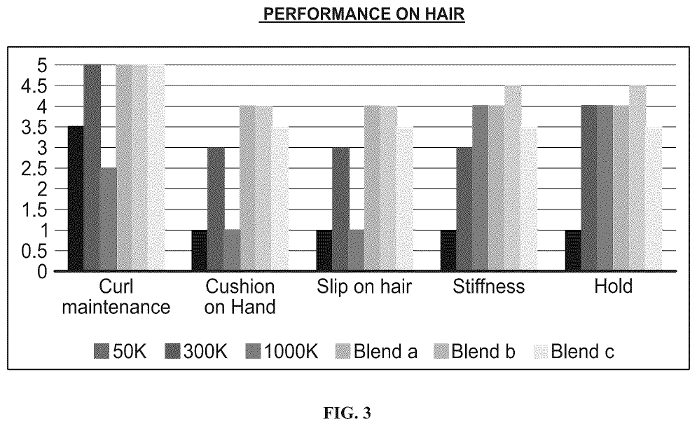

FIG. 3 depicts improved on hair performance using inventive blends (blend a, b, c) than individual samples. These multimodal compositions enable improved curl maintenance, variations of hold and stiffness, while delivering improvement in the delivery aesthetics such cushion in the and slip through the hair.

### Example 53: Hair Split Ends Mending

The split end mending benefit was evaluated using water-based solutions in which the blends are dissolved in the aqueous carrier. European, 6″ long, 1″ wide, 6.5 g net wt. medium brown, medium density virgin hair was used from International Hair Importers & Products. Hair was bleached with Wella Blondor bleaching powder and 40V developer for 35 minutes at 40° C. Hair was rinsed and washed with 12% active SLES-2EO solution and air dried. Split ends were generated by brushing and blow drying the hair for 2 hours. Hair tresses were examined periodically under a magnifying glass to confirm that there were 20 split end fibers on each tress. Each split end fiber was marked and tagged. Digital pictures of each fiber were taken with the Nikon SMZ 1500 Stereomicroscope before and after treatment. Damp hair tresses were treated with 0.14 gr of polymer solution per gm of hair, massaged in, combed through, and then air dried. Split end repair was evaluated using the following scoring criteria: 1=end split, 0.5=partial split and 0=no split. After scoring and taking digital pictures, the hair tresses were combed 20 times with a fine-tooth comb, evaluated with the scoring criteria above and digital pictures taken with the stereomicroscope. Repair was calculated using the following:

$$Total mending \% after treatment = \frac{\# Split ends before treatment - \# split ends after treatment}{\# Split ends before treatment} \times 100$$

$$Total mending \% after combing = \frac{\# Split ends before treatment - \# split ends after combing}{\# Split ends before treatment} \times 100$$

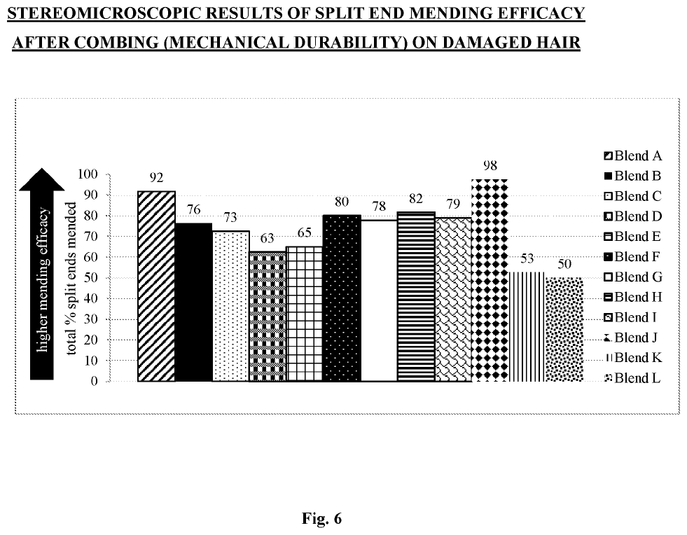

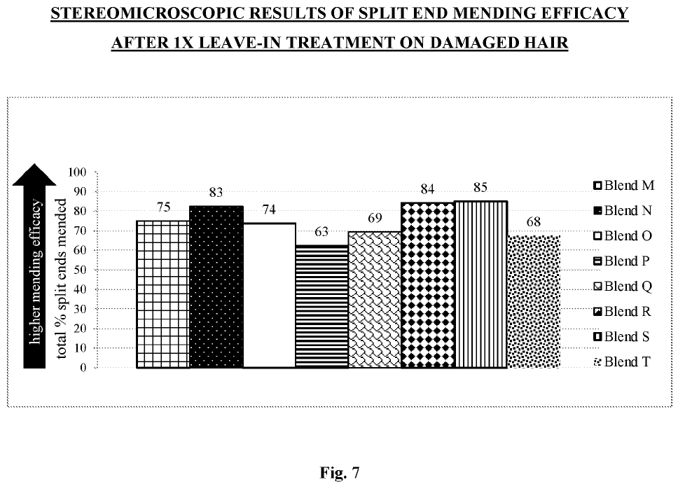

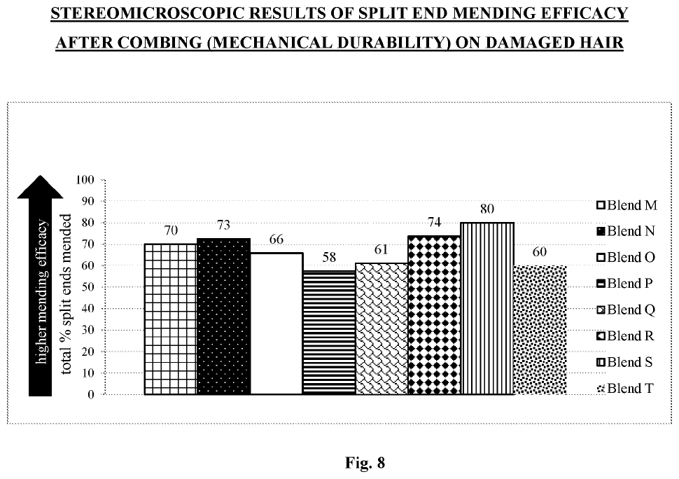

Samples from table 11 were evaluated for split end mending and results are graphed in FIGS. 5 and 6. Samples from table 12 were evaluated for split end mending and results are graphed below in FIGS. 7 and 8. FIG. 5 shows an example of a fiber during testing with a blend composition. The figures submitted along with the application demonstrate the effectiveness of the blends as a leave-in showing the percentages of repair measured before and after combing after a single treatment. All blends exhibit a positive degree of initial split end repair after one treatment and toughness against mechanical stress (combing). The higher the percent split end mending the better the efficacy.

### Example 54: Protection of Hair from Thermal & Mechanical Damage

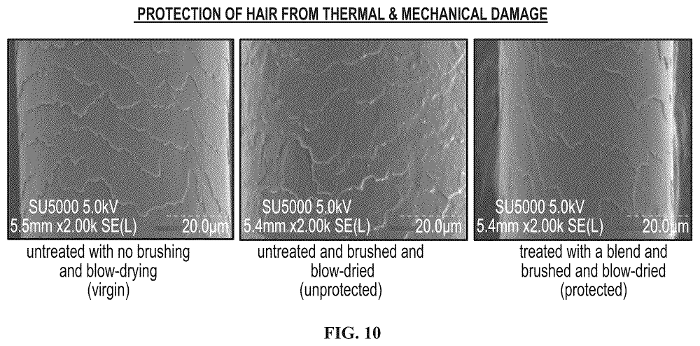

Fine density European, virgin, dark brown, hair tresses (6″ long, 1.75″ wide, 3.5 net wt.) from IHIP was used to evaluate the thermal/mechanical protection provided by the polymer blends. Hair was washed with 12% active SLES-2EO solution, rinsed, and then treated with 0.14 gr of polymer solution per gr of hair, massaged, and combed through for uniform distribution. The hair was brushed and blow-dried for 3 minutes using a 2-in-1 hair dryer brush on high heat at approximately 120° C. The hair washing, treatment, and drying cycle was repeated for a total of 7 times. After 7 cycles, hair fibers from each tress were extracted and analyzed via Scanning Electron Microscopy (SEM). Images were collected at 2000× magnification for each fiber at equidistant intervals along the fiber. FIG. 10 is an example of a blend composition from Table 12 illustrating thermal/mechanical protection from brushing and blow drying when compared to the untreated, brushed, and blow-dried control. Significant cuticle lifting, fragmentation, and removal was observed without the blends.

### Example 55: Protection of Hair from Heat Due to Hot Flat Ironing

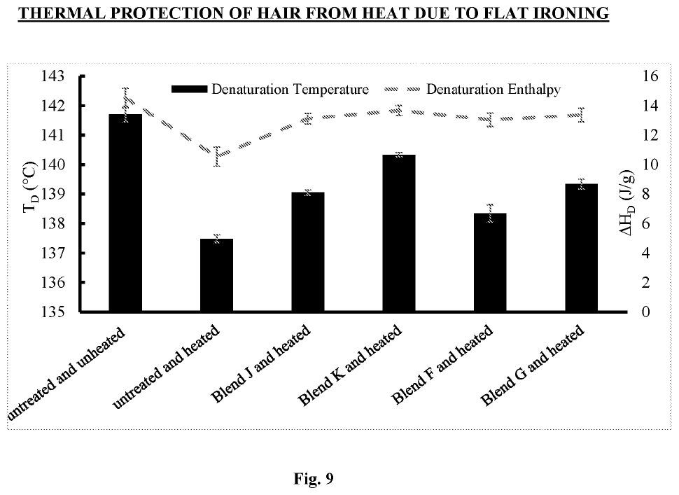

European, virgin, dark brown, fine density, 6″ long, 1.75″ wide, 3.5 g net wt. hair tresses from International Hair Importers & Products was used. Hair was washed with 12% active SLES-2EO solution, rinsed, and treated with 0.14 gm of polymer solution per gram of hair, massaged for uniform distribution and then blow-dried on cool setting until dry. The flat iron was set to 450° F. Accuracy was ensured with a digital multimeter/infrared thermometer. The flat iron was pulled from top to bottom of the tress in 3 seconds and repeated for a total of 4 minutes of heat exposure. Single fibers were taken from each tress and analyzed via Differential Scanning calorimetry (model Q2000 from TA Instruments). The thermal protective compositions of the invention are water-based solutions in which the blend is dissolved in the aqueous carrier. The present invention is an improvement in the process of styling hair where the presented compositions can protect hair from damage caused by heat insult. Protein degradation resulting for heat exposure results in reduced denaturation temperature and denaturation enthalpy. Higher TD and ΔHD is indicative of less damage. The denaturation temperature (TD) and denaturation enthalpy (ΔHD) of hair treated with Blends F, G, J, and K as seen in FIG. 9 demonstrates an improvement over the untreated and heated hair sample providing protection to the hair cortex.

Sample test conditions: 2° C./min; 25-160° C.; 10 mg snippets in 50 μL H2O; n=5. Results are tabulated below and graphically represented in FIGS. 9 & 11.

| | denaturation | | denaturation | |
|---|---|---|---|---|
| | temperature | std | enthalpy | std |
| untreated | 141.84 | 0.27 | 13.63 | 0.60 |
| untreated and | 124.28 | 0.78 | 5.42 | 0.21 |
| heated | | | | |
| Table 19 | 137.03 | 0.34 | 11.55 | 0.36 |

Therefore, we observed that, the cationic guar blends exhibit wide range of tribological behavior as compared to the individual samples as well as offer the ability to optimize use levels, weight ratios and mw/ds to deliver similar frictional behavior as commercial controls. Further, inventive composition comprising cationic guar blends is advantageous over the prior art compositions in that the composition:
(i) is versatile, works in different forms such as in a cream rinse, or in a gel form or leave in system;
(ii) helps in improving sensory attributes on hair performance characteristics such as stiffness; shine, humidity resistance;
(iii) shows positive degree of initial split end repair after one treatment and toughness against mechanical stress (combing); and
(iv) provides protection to hair cortex from thermal damage when hair is subjected to heat as evident from higher denaturation temperature (TD) and ΔHD (denaturation enthalpy) values.

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

What is claimed is:

1. A multi-modal hair care composition comprising:
(i) 0.1 to 99 wt. % of at least one cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; and
(ii) 0.1 to 99 wt. % of at least one cosmetic ingredient.

2. The hair care composition according to claim 1, wherein the composition optionally, further comprises 0.0 to 99 wt. % of a non-ionic guar.

3. The hair care composition according to claim 1, wherein the cationically modified guar blend (i) (a) has a weight average molecular weight in the range of from about 200 to about 500 KDa; and (i) (b) has a weight average molecular weight in the range of about 800 to about 1200 KDa.

4. The hair care composition according to claim 1, wherein
(A) the weight average molecular weight of cationically modified guar blend (i) (a) and (i) (b) are present in weight ratio of 92:8, 80:20, 75:25, and 60:40; and
(B) the degree of substitution (DS) of cationically modified guar blend (i) (a) or (i) (b) are present in ratio of 0.01 to 0.50.

5. The hair care composition according to claim 4(B), wherein the degree of substitution of cationically modified guar blend (i) (a) or (i) (b) are present in ratio of 0.01 to 0.10, and 0.1 to 0.20, 0.2 to 0.30, and 0.3 to 0.4.

6. The hair care composition according to claim 1, wherein the hair care composition is bi-modal, tri-modal, quadra-modal or penta-modal blend of cationically modified guars.

7. The hair care composition according to claim 1, wherein the cationically modified guar blend is selected from the group consisting of cationic Hydroxy propyl (HP) guar, Guar hydroxypropyltrimonium chloride, and cassia hydroxypropyltrimonium chloride.

8. The hair care composition according to claim 2, wherein the non-ionic guar is selected from the group consisting of non-ionic hydroxymethyl guar, hydroxyethyl guar, Hydroxypropyl (HP) guar and hydroxybutyl guar.

9. The hair care composition according to claim 1, wherein the cationically modified guar blend is selected from the group comprising:
(i) blend of (a) guar hydroxypropyltrimonium chloride having a molecular weight in the range of from about 50 to about 500 KDa, and (b) guar hydroxypropyltrimonium chloride having a molecular weight in the range of from about 800 to about 1200 KDa; and degree of substitution (DS) in the range of 0.01 to 0.3, and
(ii) blend of (a) guar hydroxypropyltrimonium chloride, and (b) cationic hydroxypropyl guar.

10. The hair care composition according to claim 1, wherein the at least one cosmetic ingredient is selected from the group consisting of acids, amino acids, antidandruff agents, anti-statics, antioxidants, anti-inflammatory agents, anti-microbial agents, acidifying agents, basifying agents, bodying agents, buffers, bleaches, cationic surfactants, cleansing agents, chelating agents, collagen, conditioning ingredients, compressed gases, dyes, de-ionized water, defoamers, emollients, emulsifiers, flexibility enhancers, fatty substances, fragrances, fillers, foam stabilizers, functional polymers, gel formers, humectants, α-hydroxycarboxylic acids, hydrophilic active agent, hair conditioning ingredients, hydrotropes, keratin hydrolyzates, light protection agents, lipophilic active agent, lipids, moisturizers, neutralizing agents, oxidizing agents, organosilicones, protein hydrolyzates, hydrotropes, perfumes, pigments, preservatives, propellants, proteins, pH modifier, polysaccharides, polyalklylene glycols, reducing agents, rheology modifiers, solvents, solubilizers, suspension agents, silicones, silicone-containing polymers, split modifiers, shine enhancers, softeners, sequestering agents, surfactants, styling polymers, thickeners, texturizing agents, tinting agents, tanning agents, UV-A or UV-B blocker/filters, UV protectants, viscosifiers, vitamins, water proofing agents, water softening agents, wetting agents, and blends thereof.

11. The hair care composition according to claim 1, wherein the composition is used for (i) hair styling, (ii) hair straightening, (iii) hair split end mending, (iv) hair cuticular smoothing, (v) hair conditioning, (vi) protecting hair from thermal and mechanical damage, and (vii) protecting hair from thermal damage from heat styling tools up to 450° F.

12. The hair care composition according to claim 1, wherein the pH of the hair care composition is in the range of from about 3 to about 13.

13. The hair care composition according to claim 1, wherein the composition is formulated as an aerosol, a cream, an emulsion, a gel, a lotion, a mousse, a powder, a paste, a solution, spray, a shampoo, a vesicle dispersion, or a wax.

14. The hair care composition according to claim 1, wherein the composition is an aerosol, a mousse, or an actuated hair spray.

15. The hair care composition according to claim 1, wherein the hair care composition used in a hair care formulation is in the range of about 0.01 wt. % to about 10.0 wt. %, or from about 0.01 to about 5.0 wt. % based on the total weight of the hair care formulation.

16. The hair care composition according to claim 10, wherein the surfactant is selected from the group consisting of amphoteric surfactants, cationic surfactants, anionic surfactants, nonionic surfactants, zwitterionic surfactants, and mixtures thereof.

17. The hair care composition according to claim 16, wherein the cationic surfactant is selected from the group consisting of cetrimonium chloride, cetrimonium bromide, dicetyldimonium chloride, and palmitamidopropyl trimonium chloride.

18. The hair care composition according to claim 1, wherein the composition exhibits a high humidity curl retention of about 80% to about 100% over a 24-hour period at 90% humidity and at 80° F.

19. The hair care composition according to claim 1, wherein the composition delivers a wide range of tribological behavior; exhibits improved humidity resistance, stiffness, durability of hold, low slip, foam enhancement and curl maintenance; is nature derived and is biodegradable.

20. A hair care composition for mending split ends comprising (i) 0.1 to 99 wt % of at least one cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; and (iii) 0.1 to 99 wt. % of at least one cosmetic ingredient.

21. The hair care composition according to claim 20, wherein the hair care composition is a leave-in composition.

22. The hair care composition according to claim 20, wherein the hair care composition is a bi-modal hair care composition.

23. The hair care composition according to claim 20, wherein the hair care composition comprises Guar hydroxypropyltrimonium chloride and Hydroxypropyl Guar Hydroxypropyl trimonium Chloride blend.

24. An aqueous hair mousse composition comprising:
(i) 0.1 to 10 wt. % of hair care composition of claim 1;
(ii) 1.0 to 60 wt. % of at least one solvent;
(iii) 0.1 to 10 wt. % of at least one cosmetic functional additive; and
(iv) 0.1 to 95 wt. % of water.

25. The mousse composition according to claim 24, wherein the at least one cosmetic functional ingredient is selected from the group consisting of acids, amino acids, antidandruff agents, anti-statics, antioxidants, anti-inflammatory agents, anti-microbial agents, acidifying agents, basifying agents, bodying agents, buffers, bleaches, cationic surfactants, cleansing agents, chelating agents, collagen, conditioning ingredients, compressed gases, dyes, de-ionized water, defoamers, emollients, emulsifiers, flexibility enhancers, fatty substances, fragrances, fillers, foam stabilizers, functional polymers, gel formers, humectants, α-hydroxycarboxylic acids, hydrophilic active agent, hair conditioning ingredients, hydrotropes, keratin hydrolyzates, light protection agents, lipophilic active agent, lipids, moisturizers, neutralizing agents, oxidizing agents, organosilicones, protein hydrolyzates, hydrotropes, perfumes, pigments, preservatives, propellants, proteins, pH modifier, polysaccharides, polyalklylene glycols, reducing agents, rheology modifiers, solvents, solubilizers, suspension agents, silicones, silicone-containing polymers, split modifiers, shine enhancers, softeners, sequestering agents, surfactants, styling polymers, thickeners, texturizing agents, tinting agents, tanning agents, UV-A or UV-B blocker/filters, UV protectants, viscosifiers, vitamins, water proofing agents, water softening agents, wetting agents, and blends thereof.

26. The mousse composition according to claim 25, wherein
(i) the preservative is selected from the groups consisting of phenoxy ethanol, caprylyl glycol or mixtures thereof;
(ii) the cationic surfactant is cetrimonium chloride, cetrimonium bromide,
dicetyldimonium chloride and palmitamidopropyl trimonium chloride; and
(iii) the gas propellant is selected from the group consisting of dimethyl ether, isobutane, 1,1-difluoro ethane, tetrafluoro propene, propane, compressed air, compressed nitrogen, compressed carbon dioxide, nitrous oxide and mixtures thereof.

27. A method for repairing split ends of hair comprising contacting hair with split ends with a multi-modal hair care composition comprising: (i) 0.1 to 99 wt. % of at least one cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; (ii) 0.0 to 99 wt. % of a non-ionic guar; and (iii) 0.1 to 99 wt. % of at least one cosmetic ingredient followed by rinsing the hair with water.

28. A method for thermal and mechanical protection from brush and blow drying, comprising contacting the hair with a multi-modal hair care composition comprising: (i) 0.1 to 99 wt. % of at least one cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; (ii) 0.0 to 99 wt. % of a non-ionic guar; and (iii) 0.1 to 99 wt. % of at least one cosmetic ingredient.

29. A method of protecting hair from the heat of hot flat ironing up to 450° F. comprising contacting the hair with a multi-modal hair care composition comprising: (i) 0.1 to 99 wt. % of at least one cationically modified guar blend comprising: (a) 1 to 99 wt. % of at least one cationically modified guar having weight average molecular weight in the range of from about 50 to about 500 KDa, and (b) 1 to 99 wt. % of at least one cationically modified guar having weight average molecular weight in the range of from about 500 to about 1500 KDa; (ii) 0.0 to 99 wt. % of a non-ionic guar; and (iii) 0.1 to 99 wt. % of at least one cosmetic ingredient.
